SEQUENCE LISTING (33 sequences)

SEQ ID NO: 1 - DNA, 708 bp, Nicotiana tabacum
ggcacgaggaaacatggggcgaattaccactaaaagtcgacgattctgaagatatggtaatttatactctcttaaaggacgctcttaacgtcggatggtcgccgtttaatttcaccgccggcgaagtaaaatcggagctaatggaggaggaaattgtggtttctccagcggatacaacggccgcgccggcggctgagttaccgaggggaaggcattacagaggtgttagacgacggccttgggggaaatttgcggcggagattagggatccggcgaagaatggagctagggttttggcttggaacatacgaaacagatgaagatgctgcaattgcttatgataaagccgcttatagaatgcgtggttcaaaggctcatttaaattttccacataggatcggtttaaatgaaccggaaccggttcgagttacggcgaaaagacgagcgtcgcctgaaccggctagttcgtcggaaaatagttcagtaaaacggagaagaaaagctgttgcagctgagaaatctggagctgtagaagtggagagtaaatcaaatgttttacaagttggatgtcaagttgaactattgacacgcggacatcaactattagtcagttaagtatgagcttacgaagattaatattcctttgtggtaatcttgcgctccgaagttgtacagtttgattttcatgttaattactctttccggtta

SEQ ID NO: 2 - DNA, 437 bp, Nicotiana tabacum
tggattgtgcgatgtataaggttttaaccaagggtaacagaggcatccaaagactcttagcttgtgtggattgggatcacgttggaagagattctgaaatgccgatgtgcctttaaggagaggggtagggagtcgattgatgaggtagactgttgtttggattgcgagggaccaaaacttgataggtacagaagcatgatgaaggatggttagcgttgtttcaataatgtggcggtatttacgatcagccgaaccaacaagttgaggggtgtatggtggacttataagatgttgaattccagaagcttgaagaattaattgtaggccaatgtactcactacctccatcagtatatgatgtgctaatggtggtggaaaagtaattagtgactaaatttttaaaattggtgaatatatggataacatcagatttatgcc

SEQ ID NO: 3 - DNA, 174 bp, Nicotiana tabacum
tgcaatcgatgagagaggtcggcttaattagctgacgagacgcctttacgtccaatcagtaggttagaagttcgaatcacatgaactgatagagttaatcaaaatgtatgtttatgtaatagcaccatctacatcttttgttgtgaatataataaaatttgtttttcttgagca

SEQ ID NO: 4 - DNA, 378 bp, Nicotiana tabacum
ttgacaataacaaagacaatgaaagttgatcattttatccggcatcttctcctttccttgcagtgccaaatctaagccatcctgaattaggatagcttccatctttaattgccacattcagaagtttgcacttcggtcaaatttctcaacataagactttgttagagtcattttggctatttaaactaacccgattagatctggctctgataccaatttgttaggatcgggaacccgggtagtgcaaaatttagccaaacaacggtataatgacaataacaaagacaatgaaagttaataataacggcaattaaagaagataaagaagacacaaatttaatatggttcggtcaaggtgacctacgtccacaagcggtc

SEQ ID NO: 5 - DNA, 283 bp, Nicotiana tabacum
taggtgaccgtgcctgagagcctgggatcatggacaaatagaatgctctttcatatgcttcgtcaccgcatctcatgatcttttctcgcagtccaatcatcttgtaagattgttataataaagaaacctatttgtcttttgtttattcaagttttgtgaagtttgaaataagtttcaccactatcgagattgaaacacccttcctgtatctgctgcattcaagttcccgatcatgtgaagtgctttctttccgagttataaagcttttacatctttcttgcca

SEQ ID NO: 6 - DNA, 379 bp, Nicotiana tabacum
ttgacaataacaaagacaatgaaagttgatcattttatccggcatcttctcctttccttgcagtgccaaatctaagccatcctgaattaggatagcttccatctttaattgccacattcagaagtttgcacttcggtcaaatttctcaacataagactttgttagagtcattttggctatttaaactaacccgattagatctggctctgataccaatttgttaggatcgggaacccgggtagtgcaaaatttagccaaacaacggtataatgacaataacaaagacaatgaaagttaataataacggcaattaaagaagataaagaagacacaaatttaatatggttcggtcaaggtgacctacgtccacaagcggtca

SEQ ID NO: 7 - DNA, 204 bp, Nicotiana tabacum
tcgtcagtgacaaatgggtctagcagctctgaggtattccaagaattggaggcaccatgctcttctggacaacctcaaattctccagctctttcccgagcattcatgatttagaatcacttgcagcctctgttctagcgaaaaggcaaaaaatggccggatgtcaacgttgacgtgaagattttggatattatgttttcctgca

SEQ ID NO: 8 - DNA, 299 bp, Nicotiana tabacum
tgaggagaagaaagaggctaaggtgattagtatttctgaggaagggaatgccgctgaagatattaaagctactaaagctgcaatgtaaacaagtgcacatgtgatattttgtttaagatcttttgttttttccccatcgcctttgttgttgtgagttaatttagtttctgtgaatgaaacttatgtcatgtatccattttctgtatgtaatttaatgtcgtacgaaaaatatgatcagtgtaatagattgtttttctgtaagtgtaagatcaaattaataaaagagttgattcagtgca

SEQ ID NO: 9 - DNA, 276 bp, Nicotiana tabacum
ttgggaaaagaaagaaatccttataaaaactcggtctttacaatttactgactaatagaagatgcatgtttacacggtgcatgaaggttgctgcgaatgagtttgtactgatgtacatgggttcttttgtaagaattctgacttctgatggttgggaaacgtatcaggctaatataagggaaacatatttgaaagagtaaagcaataaattacataagataagacaagagaagatttatgtggttcggcaatttttgtctactccacggtcaccta

SEQ ID NO: 10 - DNA, 2169 bp, Nicotiana tabacum
  CDS (525)..(1955)
  unsure (1951): Replace 1951, “g”
  prim_transcript (432)..(2049)
gttaaaactttaaacaaagttttcgtgtttgaacagaacactggtcttaaaggtaacgtttttggctttttttgagtctgcgctcgacttgtgagttgtgacggaagagcatatgtcacattcatcatctgtattcgtacaatgccctcttctctaagaaattgggagtcaccacccctaggcgcccaccactttatcttcattttatgattggctctatgtctttgcctgttaaatcaatcacgcgagtgcttacgtacgtactctgattcaaccaaataattgaaaaactttttccttgaagaaaagtcagtcttaactcatactaatatgtagcttcaacgaaagtcacaactaattccacgtataaataaagtgagagtactctcgtaactgcagcattaaaaaacactcatgcaatcatacaagaactgaaaacaaccacacagctttatatttctttctattttctgctactaaactaggagtacatctttctttctttctttcaagcattttcacaaatgggtcagctccatattttcttctttcctgtgatggctcatggccacatgattcctacactagacatggcgaagctctttgcttcacgtggtgttaaggccactataatcacaaccccactcaatgaattcgttttctccaaagctattcaaagaaacaagcatttgggtatcgaaatcgaaatccgtttgatcaaattcccagctgttgaaaacggcttacctgaagaatgcgaacgcctcgatcaaatcccttcagatgagaagctcccaaactttttcaaagctgtagctatgatgcaagaaccactagaacagcttattgaagaatgtcgccccgattgtcttatttcagatatgttccttccttggactactgatactgcagcaaaatttaacattccaagaatagtctttcatggcacaagcttctttgctctttgtgttgagaatagcgtcaggctaaataagcctttcaagaatgtgtcctcagattctgaaacttttgttgtaccggatttgcctcacgaaattaagctgaccagaacccaggtgtctccgtttgagcgatctggggaagagacggctatgacccggatgataaaaacagtcagggaatcagattcaaagagctatggagttgttttcaacagtttctatgagcttgaaacagattatgttgagcattatactaaggtgctgggtagaagagcttgggctattggccctctatcgatgtgcaacagggacattgaagataaagctgaaagaggaaagaaatcctctattgataaacacgagtgcttgaaatggcttgattcgaagaaaccaagttccgtcgtttacatttgttttggaagcgtagcgaatttcactgcatcacaactgcacgaacttgctatgggagttgaagcttccggacaagaattcatttgggttgttagaacagaactagacaacgaagattggttgcctgaaggattcgaggaaagaacgaaagagaaaggtttaataataagaggatgggcaccccaagtactaattcttgatcacgaatctgtgggagcttttgttacacattgtggttggaattcaacactagaaggagtttcaggaggggttccaatggtaacatggcctgtatttgctgagcaatttttcaatgagaagttagtgactgaggttttgaaaactggagctggtgttggttcgatacaatggaagagatcagctagtgaaggagtgaaaagagaagcaatagctaaggcaataaagagagtaatggtgagtgaagaagcagatggattcagaaacagagctaaagcgtataaggagatggcaagaaaggctattgaagaaggagggtcatcttacactggattgactactttgttggaagatataagtacatatagttccactggtcattaagttatgaatagcaacaaaaaaaaatgtagtactccgtacttggtattatttctgtactgtttttgtgcttttcctgtatgtgctaatttaaacatttctttttgtcactttttataattgcaatgatgatgatatgtggaagaaccccttgcctccatttctcctttatttaggatagtaagattattttcagccgaaaatgagatccggatcc

SEQ ID NO: 11 - protein, 476 aa, Nicotiana tabacum
MGQLHIFFFPVMAHGHMIPTLDMAKLFASRGVKATIITTPLNEFVFSKAIQRNKHLGIEIEIRLIKFPAVENGLPEECERLDQIPSDEKLPNFFKAVAMMQEPLEQLIEECRPDCLISDMFLPWTTDTAAKFNIPRIVFHGTSFFALCVENSVRLNKPFKNVSSDSETFVVPDLPHEIKLTRTQVSPFERSGEETAMTRMIKTVRESDSKSYGVVFNSFYELETDYVEHYTKVLGRRAWAIGPLSMCNRDIEDKAERGKKSSIDKHECLKWLDSKKPSSVVYICFGSVANFTASQLHELAMGVEASGQEFIWVVRTELDNEDWLPEGFEERTKEKGLIIRGWAPQVLILDHESVGAFVTHCGWNSTLEGVSGGVPMVTWPVFAEQFFNEKLVTEVLKTGAGVGSIQWKRSASEGVKREAIAKAIKRVMVSEEADGFRNRAKAYKEMARKAIEEGGSSYTGLTTLLEDISTYSSTGH

SEQ ID NO: 12 - DNA, 19 bp, Artificial Sequence
  Description of Artificial Sequence:PCR Primer
attaccctcactaaaggga

SEQ ID NO: 13 - DNA, 26 bp, Artificial Sequence
  Description of Artificial Sequence:PCR Primer
gggaagcttctgcagttacgagagta

SEQ ID NO: 14 - DNA, 26 bp, Artificial Sequence
  Description of Artificial Sequence:PCR Primer
ccgaagcttagctgacccatttgtga

SEQ ID NO: 15 - DNA, 10 bp, Artificial Sequence
  Description of Artificial Sequence Primer AP1
agccagcgaa

SEQ ID NO: 16 - DNA, 10 bp, Artificial Sequence
  Description of Artificial Sequence:Primer AP2
gaccgcttgt

SEQ ID NO: 17 - DNA, 10 bp, Artificial Sequence
  Description of Artificial Sequence:Primer  AP3
aggtgaccgt

SEQ ID NO: 18 - DNA, 10 bp, Artificial Sequence
  Description of Artificial Sequence:Promer AP4
ggtactccac

SEQ ID NO: 19 - DNA, 10 bp, Artificial Sequence
  Description of Artificial Sequence:Primer AP5
gttgcgatcc

SEQ ID NO: 20 - DNA, 10 bp, Artificial Sequence
  Description of Artificial Sequence:Primer AP6
gcaatcgatg

SEQ ID NO: 21 - DNA, 10 bp, Artificial Sequence
  Description of Artificial Sequence:Primer AP7
ccgaaggaat

SEQ ID NO: 22 - DNA, 10 bp, Artificial Sequence
  Description of Artificial Sequence:Primer AP8
ggattgtgcg

SEQ ID NO: 23 - DNA, 10 bp, Artificial Sequence
  Description of Artificial Sequence:Primer AP9
cgtggcaata

SEQ ID NO: 24 - DNA, 10 bp, Artificial Sequence
  Description of Artificial Sequence:Primer AP10
tagcaagtgc

SEQ ID NO: 25 - DNA, 10 bp, Artificial Sequence
  Description of Artificial Sequence:Primer AP11
cagaccgttc

SEQ ID NO: 26 - DNA, 10 bp, Artificial Sequence
  Description of Artificial Sequence:Primer AP12
tgctgacctg

SEQ ID NO: 27 - DNA, 10 bp, Artificial Sequence
  Description of Artificial Sequence:Primer AP13
agttaggcac

SEQ ID NO: 28 - DNA, 10 bp, Artificial Sequence
  Description of Artificial Sequence:Primer AP14
aatgggctga

SEQ ID NO: 29 - DNA, 10 bp, Artificial Sequence
  Description of Artificial Sequence:Primer AP15
agggcctgtt

SEQ ID NO: 30 - DNA, 10 bp, Artificial Sequence
  Description of Artificial Sequence:Primer AP16
cgtcagtgac

SEQ ID NO: 31 - DNA, 10 bp, Artificial Sequence
  Description of Artificial Sequence:Primer AP17
gcaaggagtc

SEQ ID NO: 32 - DNA, 10 bp, Artificial Sequence
  Description of Artificial Sequence:Primer AP18
ctgagctagg

SEQ ID NO: 33 - DNA, 10 bp, Artificial Sequence
  Description of Artificial Sequence:Primer AP19
ggctaatgcc

(19) United States
(12) Patent Application Publication
(10) Pub. No.: US 2002/0039780 A1
(43) Pub. Date: Apr. 4, 2002

(54) Salicyclic acid inducible genes and promoters

(76) Inventors: Diana M. Horvath, New York, NY (US); Nam-Hai Chua, Scarsdale, NY (US); Maarten H. Stuiver, Leiden CB (NL); Ian Jepson, Berkshire (GB)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 09/777,207

(22) Filed: Feb. 5, 2001

Related U.S. Application Data
(63) Continuation of application No. PCT/EP99/05581, filed on Aug. 2, 1999.
(60) Provisional application No. 60/095,187, filed on Aug. 3, 1998.

(51) Int. Cl. C12P 21/02; C12N 5/04; C07H 21/04; C12N 9/50

(52) U.S. Cl. 435/219; 435/410; 435/320.1; 435/69.1; 536/23.2

(57) ABSTRACT

The invention describes novel nucleotide sequences from genes which are produced upon induction with salicylic acid in tobacco plants. The genes can be used to confer resistance to pathogens in susceptible plants. Another part of the invention is formed by the promoters regulating expression of these genes. These promoters are switched on early in the response to pathogen attack and can be used as pathogen-inducible promoters.

### FIELD OF THE INVENTION

The invention is directed to the field of pathogen resistance in plants, specifically through new salicylic acid inducible genes and promoters, vectors, hosts and cells harboring same. The invention further relates to plants incorporating these genes, and to plants which as a result thereof show reduced susceptibility to fungal pathogens. Further, the invention is directed to the use of the promoters as pathogen-inducible promoters to drive expression of genes which can confer pathogen resistance to the plant hosts. Also, the invention is directed to use of the promoters to induce expression of phenotypic traits as a result of salicylic acid (SA) or SA-homologues treatment, other than normally induced by SA.

### BACKGROUND ART

When a host plant is attacked by viral, bacterial, or fungal pathogens, it responds in many ways. It is assumed that most of the responses are designed to protect the plant by eliminating or restricting the pathogen and limiting the damage that the pathogen causes. In some cases the defense attempts are unsuccessful, and the pathogen replicates and spreads throughout the plant, usually causing damage or even death of the host. Even in those cases that a pathogen successfully infects, plants raise a defense response, but generally too small in magnitude or too slow.

Two mechanisms in which plants build resistance against attacking pathogens have been studied extensively. These are the hypersensitive response (HR) and the systemic acquired resistance (SAR). The HR is, under natural conditions, very specific and confined to relations between a specific pathovar and a specific plant variety, which seem to follow the gene-for-gene relationship (Flor, Ann. Rev. Phytopathol. 9: 275-296, 1971). This is, single dominant genes of the pathogen elicit a hypersensitive response on plants carrying the corresponding dominant resistance gene.

As a consequence of the hypersensitive response, plants enter into a state of increased defense capabilities, which is called SAR (systemic acquired resistance) and protects the plant from infections with otherwise virulent pathogens.

The signalling pathways involved in both the development of the hypersensitive response and SAR are poorly understood. Early studies have shown that salicylic acid (SA) accumulates to substantial levels both locally and systemically. Also, treatment of plants with salicylic acid increases the level of expression of PR-genes, and increases the resistance of plants to pathogens suggesting that SA plays a crucial role in the establishment of SAR (see e.g. Ryals, Plant Cell 8, 1809-1819, 1996).

Even more firm evidence for the crucial role of SA was obtained by making transgenic plants carrying the nahG gene from Pseudomonas putida. The geneproduct of the nahG gene hydroxylates SA, and renders it inactive. NahG-transgenic tobacco and Arabidopsis thaliana plants are compromised in their ability to raise an effective hypersensitive response, since the pathogen grows and spreads from the initial infection site (Gaffney et al., Science 261, 754-756, 1993; Delaney et al., Science 266, 1247-1250, 1994). The nahG-transgenic plants are also defective in raising a SAR response.

It is, however, clear that alternative pathways for induction of the hypersensitive response are present, and overexpression of nahG in tomato does not compromise the hypersensitive response occurring after challenge of Cf9 or Cf2 plants with Cladosporium fulvum races containing Avr9 and Avr2, respectively. (Hammond-Kosack & Jones, Plant Cell 8, 1773-1791, 1996).

Overviews of the role of SA in plant disease signalling can be found in Durner et al. (Trends. Plant. Sci. 2, 266-274, 1997); Chasan (Plant Cell 7, 1519-1521, 1995); Klessig & Malamy (Plant Mol. Biol. 26, 1439-1458, 1994); and Malamy, J. and Klessig, D. A. (The Plant J., 2, 643-654, 1992).

The state of SAR has been shown to correspond with the coordinate expression of at least 16 genes in tobacco (Ward et al., Plant Cell 3: 1085-1094, 1991), including those encoding the PR-proteins (reviewed in Bol, J. F. and Van Kan, J. A., Microbiol. Sci. 5(2), 47-52, 1988, and Bowles D. J., Annu. Rev. Biochem. 59, 873-907, 1990).

Some progress has been made in identifying signal transduction components that mediate the SA response. An SA-binding protein identified as catalase was initially thought to convert the SA signal into increases in hydrogen peroxide (H₂O₂), by inhibition of catalase function (Chen, et al., 1993, Science, 262: 1883-1886). Because H₂O₂ treatment was found to induce PR gene expression in tobacco, Klessig and co-workers proposed that H₂O₂ was downstream signal in the pathway to induced defense responses. Subsequently, however, it has been demonstrated that H₂O₂ induction of PR genes is dependent on SA accumulation (Bi, et al., 1995, Plant J., 8: 235-245; Neuenschwander, et al., 1995, Plant J., 8: 227-233). Recently, a second SA binding protein SABP2, has been characterised with a 150-fold higher binding affinity than catalase (Du and Klessig, 1997, Plant Physiol., 113: 1319-1327). Several features of SABP2 are consistent with this protein functioning as an SA receptor. Jupin and Chua (1996, EMBO J., 15: 5679-5689) have identified a tobacco DNA-binding activity, known as SARP, that consists of a TGA1a-related bZIP-family transcription factor. In the absence of SA, SARP exists as an inactive complex, but treatment with SA for 1 h produced binding to the SA-inducible as-1 element of the Cauliflower Mosaic Virus (CaMV) 35S promoter. SARP could be released from its inhibitory component, SAI, by treatment of control extracts with dissociating agents. Because phosphatase treatment of SA-induced extracts blocked DNA binding activity, it is likely that the natural signal transduction pathway to release SARP following SA treatment involves phosphorylation. One kinase with features consistent with this response is the SIP kinase, a MAP kinase family member that is rapidly and transiently induced following treatment of cultured tobacco cells with SA. Kinase activity increased from background to peak activity with 5 min. and diminished by 45 min. (Zhang, and Klessig, 1997, Plant Cell, 9: 809-824). Finally, the NPR1/NIM1/SAI1 protein is an ankyrin-repeat/IkB-like protein that is a positive regulator of SA perception or PR gene induction (Cao, et al., 1997, Cell, 88: 57-63; Ryals, et al., 1997, Plant Cell 9: 425-439); Shah, et al., 1997, Mol. Plant-Microbe Interact., 10: 69-78). Arabidopsis plants deficient for this function fail to induce PR genes in response to SA treatment or pathogen attack and are more susceptible to disease. Identification of these components has helped tremendously to understand early SA signal transduction. Collectively, they form a model in which a receptor relays increases in SA to latent signalling components, such as kinases, which modify transcription factors that target promoters in SA-induced genes.

In contrast, little progress has been made on the identification of SA-response genes. The best characterised are the PR genes, which are induced to high levels following treatment with incompatible pathogens or direct application of SA (Eyal, et al., 1992, Plant Mol. Biol., 19: 589-599; Ward, et al., 1991, Plant Cell, 3: 1085-1094). The response of PR genes to SA is first evident at 6-8 h and rises steadily over several days. On the time scale of induced disease resistance, this response has been considered as rapid; however, on the time scale of molecular responses to a stimulus, the response is slow. It has been established that PR gene expression is contingent on prior gene induction events (Qin, et al., 1994, Plant Cell, 6: 863-874; Uknes, et al., 1993, Plant Cell, 5: 159-169).

Identification of similar primary genetic responses of mammalian cells to cytokine induction (Almendral, et al., 1988, Mol. Cell. Biol., 8: 2140-2148; Beadling, et al., 1993, Proc. Natl. Acad. Sci. USA, 90: 2719-2723; Larner, et al., 1986, J. Biol. Chem., 261: 453-459; Lau and Nathans, 1987, Proc. Natl. Acad. Sci. USA, 84: 1182-1186; Zipfel, et al., 1989, Mol. Cell. Biol., 9: 1041-1048) has revealed scores of genes that have provided a foundation for extensive advances in the understanding of these signalling pathways over the past ten years. We set out to identify early SA response genes, which may include transcription factors or signal transduction components for PR gene expression, to learn more about how SA functions at the molecular level. Identification of such genes would make it possible to characterise events involved in the establishment of later responses during the induction of disease resistance. They would also provide an opportunity to characterise the cis-elements, transcription factors and upstream signalling components involved in the primary responses of cells to SA. Identification factors and upstream signalling components involved in the primary responses of cells to SA.

### SUMMARY OF THE INVENTION

The invention comprises a polynucleotide which when opereably linked to its native regulatory sequence is capable of producing a protein upon induction with salicylic acid characterised in that the said polynucleotide comprises a sequence which is selected from the group essentially consisting of SEQ ID Nos. 1 to 9 or is a polynucleotide sequence which encodes the amino acid sequence of SEQ ID No. 11. The invention also comprises chimeric DNA sequences comprising such a nucleotide sequence, preferably further comprising a transcriptional initiation region and, optionally, a transcriptional termination region. Especially preferred are chimeric DNA sequences wherein the transcriptional initiation region is an inducible promoter, the inducible promoter being a pathogen inducible promoter or a chemically inducible promoter.

Such a chimeric DNA sequence can be situated in a vector.

Further part of the invention are host cells comprising such a vector which are capable of maintaining said vector once present therein and/or host cells stably incorporating in its genome a nucleotide sequence as defined above.

An other part of the invention is a promoter characterised in that it comprises the nucleic acid sequence naturally occurring 5′ and capable of regulating the transcription of the polynucleotide or polynucleotide sequence as defined above. Preferably such a promoter comprises nucleotides 1-431 of SEQ ID NO: 10.

Further part of the invention is a vector comprising a pathogen inducible promoter according to the invention and host cell comprising such a vector.

The host cells of the invention preferably are plant cells.

Further part of the invention is a plant or a plant part comprising at least one such plant cell or consisting essentially of such plant cells.

An other part of the invention is a method to make plants resistant to pathogen attack, characterized in that they are transformed with a vector comprising a nucleic acid sequence according to the invention.

Still an other part of the invention is a method for expressing proteins in plants characterised in that the promoter according to the invention is used as the regulatory region which is operably linked to a polynucleotide encoding the said protein to be expressed, specifically wherein the said promoter is induced by salicylic acid or a homologue thereof or wherein the promoter is induced by a pathogen infection.

A further embodiment of the invention is a method to induce in plants phenotypic traits other than those normally induced by SA-treatment

### DESCRIPTION OF THE FIGURES

FIG. 1. Four classes of SA-induced early genes. Northern analysis of genes identified by differential display. Gel-purified DNA fragments produced by ddPCR were either used directly for random-primed labeling and probing of Northern filters (e.g. C7-1, C6-1, and G9-2) or first subcloned and released by restriction digest. Each lane contains 1 μg of poly(A) mRNA from cultured tobacco cells treated with (1) H₂O, 4 hr; (2) 200 μM SA, 4 hr; (3) 71 μM CHX, 5 hr; (4) 71 μM CHX, 5 hr plus 200 μM SA after 1 hr (4 hr total); (5) 20 μM SA, 2 hr; (7) 200 μM SA, 8 hr. One blot was sequentially hybridized with the G1-1 fragment and the β-ATPase gene as a loading control probe. response classes (I-IV) are indicated on the left of the autoradiograms.

FIG. 2. Time course of SA-induced gene expression. Northern analysis of representative differential display genes following treatment of cultured tobacco cells with SA for the indicated time (in hours, except lanes 2-4, which refer to 10, 20, and 30 min.). Twenty micrograms of total RNA was loaded per lane. Blots were hybridized with ddPCR fragments indicated at right or with the β-ATPase gene as a loading control probe. Autoradiogram exposure times were optimized for each gene. The signal for C18-1 was enhanced by using the full length cDNA as probe. Labels on left indicate response class.

FIG. 3. Effect of CHX on time-dependent accumulation of mRNA following SA-treatment. Northern analysis of representative differential display genes following treatment of cultured tobacco cells with H₂O (lanes 1-4), 200 μM SA (lanes 5-9), 71 μM CHX (lanes 10-14), 71 μM CHX plus 200 μM SA (lanes 15-19) for 0.5, 1, 4, 8 or 24 hr (Panel A) or 0.5, 1, 3, 5, or 10 hr (Panel B). CHX pretreatments were initiated 1 hr prior to the start of each time course, thus CHX incubation times are 1 hr longer than the indicated time. Twenty micrograms of total RNA was loaded per lane. Blots were hybridized with ddPCR fragments indicated at right or with the β-ATPase geneas a loading control probe. Autoradiogram exposure times were optimized for each gene. Labels on left indicate response class.

FIG. 4. Specificity of induction among SA analogs and other signalling compounds. Northern analysis of representative differential display genes following treatment of tobacco cells with 100 μM or 1 mM (left and right side of triangles, respectively) of the following chemicals for 2-2.5 hr: salicylic acid (SA, lanes 4 and 5), acetylSA (ASA, lanes 6 and 7), benzoic acid (BA, lanes 8 and 9), 4-hydroxybenzoic (4HBA, lanes 10 and 11), thiamine (thia, lanes 12 and 13), methyljasmonate (MJ, lanes 14 and 15), abscisic acid (ABA, lanes 16 and 17), or 2,4-dichlorophenoxyacetic acid (2,4-D, lanes 18 and 19). Control treatments are H₂O (H, lane 1), 1% ethanol (E, solvent concentration in MJ treatments, lane 2), or 1% DMSO (D, solvent concentration in 2,4D treatments, lane 3). Blots were hybridized with ddPCR fragments indicated at right or with the β-ATPase geneas a loading control probe. Autoradiogram exposure times were optimized for each gene. The signal for C18-1 was enhanced by using the full length cDNA as probe. Labels on left indicate response class.

FIG. 5. Dose response of C18-1 and G8-1 to a range of chemicals. Northern analysis of tobacco cell cultures with eight concentrations of SA, ASA, BA, 4HBA, Thia, MJ, ABA, 2,4-D, or H₂O for 2-2.5 hr. Concentrations were 0.1, 1, 3, 10, 30, 100, 300 μM or 2 nM, as indicated.

FIG. 6. Sensitivity to SA concentration of several SA-induced genes. Graphic representation of mRNA accumulation of G8-1 ( ), C18se -1 (♦), and IEGT (o) in response to a 2-2.5 hr incubation of cultured tobacco cells with a range of SA concentrations. RNA gel blots were quantitated by phosphorimaging analysis and normalized relative to the β-ATPase gene loading control (not shown).

FIG. 7. Induction time course of gene expression in tobacco plants inoculated with TMV. Northern analysis of C18-1, IEGT, and G8-1 mRNA accumulation in leaves of resistant tobacco plants inoculated with TMV (+) or mock-inoculated (m) and harvested at 28, 32, 36, 40, 56, and 74 hours post-inoculation (hpi). Leaves from two uninoculated plants (−) were sampled at the start of the treatment period. Each lane represents a separate, individual plant. RNA gel blots were also probed with the Pr1a gene and the β-ATPase gene as a loading control. Labels on left indicate response class.

FIG. 8. A. Schematic representation of the set-up of the experiment investigating SA-inducible gene expression in plants carrying a promoter-luciferase reporter construct. Parts of the leaves of transgenic plants are harvested, split and one half treated by infiltration by SA. The other half is treated with water. Then both halves are sprayed with luciferin solution and tested for luminescence. B. Results of the experiment using leaves of tobacco transgenic for the IEGT/G1-1 promoter-luciferase reporter construct. On the picture, the lefthand side of the leave was infiltrated with SA, the right hand side infiltrated with water. Increased luminescence is indicated by the lighter colors.

### DETAILED DESCRIPTION OF THE INVENTION

We describe here a collection of 15 genes that are induced within 2-3 h of SA treatment. These genes show several distinct response patterns, including induction during an incompatible pathogen interaction. Identification of these genes not only significantly enhances our understanding of the primary transcriptional responses of cells to SA and provides a large number of new molecular markers for SA action, but it also gives the opportunity to use these genes to set in motion the cascade of events that are responsible for generating pathogen resistance without the trigger induced by an increased SA concentration. It is envisaged that overexpression of the genes of the invention starts the cascade leading eventually to induction of PR-genes and the development of a systemically acquired resistance. It is furthermore envisaged that a continuous expression of the gene(s), which would lead to a continuous state of acquired resistance, would hamper the plants in their natural development. A preferred embodiment of the invention would be an expression system where the gene(s) is (are) under expression of an inducible promoter, which can be switched on purposefully. Such a system would enable the plants to express the gene(s) at a timepoint or occasion when a systemic resistance would be advantageous.

It must be understood that the nucleotide sequences coding for the gene products may be changed freely as long as the resulting gene product still is able to trigger the cascade leading to acquired resistance. In particular a person skilled in the art would recognize that variants of the nucleotide sequences can be produced by accounting for the degeneracy of the genetic code or by changes to the codon usage to adapt it to the codon usage which is most similar to the plant to which the genes will be transformed. Also the polynucleotide used for transformation may be modified in that mRNA instability encoding motifs and/or fortuitous splice regions may be removed so that expression of the thus modified polynucleotides yields substantially similar gene products. Further sequences of the invention may be identified by their ability to hybridize with the sequences listed in the sequence listing under stringent conditions. Stringent conditions in this respect means a reaction at a temperature of between 60° C. and 65° C. in 0.3 strength citrate buffered saline containing 0.1% SDS followed by rinsing at the same temperature with 0.3 strength citrate buffered saline containing 0.1% SDS.

The genes of the invention encode proteins. The word protein means a sequence of amino acids connected trough peptide bonds. Polypeptides or peptides are also considered to be proteins. Muteins of the protein of the invention are proteins that are obtained from the proteins depicted in the sequence listing by replacing, adding and/or deleting one or more amino acids, while still retaining their activity, i.e. the ability to trigger the resistance cascade. Such muteins can readily be made by protein engineering in vivo, e.g. by changing the open reading frame capable of encoding the enzyme such that the amino acid sequence is thereby affected. As long as the changes in the amino acid sequences do not altogether abolish the enzymatical activity such muteins are embraced in the present invention. Further, it should be understood that mutations should be derivable from the proteins or the DNA sequences encoding these proteins depicted in the sequence listing while retaining biological activity, i.e. all, or a great part of the intermediates between the mutated protein and the protein depicted in the sequence listing should have enzymatical activity. A great part would mean 30% or more of the intermediates, preferably 40% of more, more preferably 50% or more, more preferably 60% or more, more preferably 70% or more, more preferably 80% or more, more preferably 90% or more, more preferably 95% or more, more preferably 99% or more. In particular substitutions in the sequence may be made between the following amino acid groups viz.

(a) Alanine, Serine, Glycine and Threonine

(b) Glutamic acid and Aspartic acid

(c) Arginine and Lysine

(d) Isoleucine, Leucine, Valine and Methionine

(e) Phenylalanine, Tyrosine and Tryptophan

The present invention provides a chimeric DNA sequence which comprises an expression cassette according to the invention. In this specification the term “chimeric”, with reference to a DNA sequence includes a DNA sequence comprising the open reading frame of the nucleotides mentioned above wherein the sequence is altered by fusion with another sequence whether such other sequence may be endogenous or non endogenous to the plant, also a sequence which is mutated whether by addition, deletion, substitution, wherein said chimeric DNA is formed in situ or otherwise.

Chimeric DNA shall not be limited to DNA molecules which are replicable in a host, but shall also mean to comprise DNA capable of being ligated into a replicon, for instance by virtue of specific adaptor sequences, physically linked to the open reading frame according to the invention. The open reading frame may or may not be linked to its natural upstream and downstream regulatory elements.

The open reading frame may be derived from a genomic library. In this latter embodiment it may contain one or more introns separating the exons making up the open reading frame that encodes a protein according to the invention. The open reading frame may also be encoded by one uninterrupted exon, or by a cDNA to the mRNA encoding a protein according to the invention. Open reading frames according to the invention also comprise those in which one or more introns have been artificially removed or added. Each of these variants is embraced by the present invention.

In order to be capable of being expressed in a host cell a chimeric DNA according to the invention will usually be provided with regulatory elements enabling it to be recognised by the biochemical machinery of the host and allowing for the open reading frame to be transcribed and/or translated in the host. It will usually comprise a transcriptional initiation region which may be suitably derived from any gene capable of being expressed in the host cell of choice, as well as a translational initiation region for ribosome recognition and attachment. In eukaryotic cells, an expression cassette usually comprises in addition a transcriptional termination region located downstream of said open reading frame, allowing transcription to terminate and polyadenylation of the primary transcript to occur. In addition, the codon usage may be adapted to accepted codon usage of the host of choice. Further, often a signal sequence may be encoded, which is responsible for the targeting of the gene expression product to subcellular compartments. The principles governing the expression of a chimeric DNA construct in a chosen host cell are commonly understood by those of ordinary skill in the art and the construction of expressible chimeric DNA constructs is now routine for any sort of host cell, be it prokaryotic or eukaryotic.

In order for the open reading frame to be maintained in a host cell it will usually be provided in the form of a replicon comprising said open reading frame according to the invention linked to DNA which is recognised and replicated by the chosen host cell. Accordingly, the selection of the replicon is determined largely by the host cell of choice. Such principles as govern the selection of suitable replicons for a particular chosen host are well within the realm of the ordinary skilled person in the art.

A special type of replicon is one capable of transferring itself, or a part thereof, to another host cell, such as a plant cell, thereby co-transferring the open reading frame according to the invention to said plant cell. Replicons with such capability are herein referred to as vectors. An example of such vector is a Ti-plasmid vector which, when present in a suitable host, such as Agrobacterium tumefaciens, is capable of transferring part of itself, the so-called T-region, to a plant cell. Different types of Ti-plasmid vectors (vide: EP 0 116 718 B1) are now routinely being used to transfer chimeric DNA sequences into plant cells, or protoplasts, from which new plants may be generated which stably incorporate said chimeric DNA in their genomes. A particularly preferred form of Ti-plasmid vectors are the so-called binary vectors, essentially as claimed in EP 0 120 516 B1 and U.S. Pat. No. 4,940,838. Other suitable vectors, which may be used to introduce DNA according to the invention into a plant host, may be selected from the viral vectors, e.g. non-integrative plant viral vectors, such as derivable from the double stranded plant viruses (e.g. CaMV) and single stranded viruses, gemini viruses and the like. The use of such vectors may be advantageous, particularly when it is difficult to stably transform the plant host. Such may be the case with woody species, especially trees and vines.

The expression “host cells incorporating a chimeric DNA sequence according to the invention in their genome” shall mean to comprise cells, as well as multicellular organisms comprising such cells, or essentially consisting of such cells, which stably incorporate said chimeric DNA into their genome thereby maintaining the chimeric DNA, and preferably transmitting a copy of such chimeric DNA to progeny cells, be it through mitosis or meiosis. According to a preferred embodiment of the invention plants are provided, which essentially consist of cells which incorporate one or more copies of said chimeric DNA into their genome, and which are capable of transmitting a copy or copies to their progeny, preferably in a Mendelian fashion. By virtue of the transcription and translation of the chimeric DNA according to the invention in some or all of the plant's cells, those cells that express the genes will show enhanced resistance to pathogen infections. Although the principles as indicated above governing transcription of DNA in plant cells are not always understood, the creation of chimeric DNA capable of being expressed in substantially a constitutive fashion, that is, in substantially most cell types of the plant and substantially without serious temporal and/or developmental restrictions, is now routine. Transcription initiation regions routinely in use for that purpose are promoters obtainable from the cauliflower mosaic virus, notably the 35S RNA and 19S RNA transcript promoters and the so-called T-DNA promoters of Agrobacterium tumefaciens, in particular to be mentioned are the nopaline synthase promoter, octopine synthase promoter (as disclosed in EP 0 122 791 B1) and the mannopine synthase promoter. In addition plant promoters may be used, which may be substantially constitutive, such as the rice actin gene promoter, or e.g. organ-specific, such as the root-specific promoter. Preferably, inducible promoters may be used which enable induction of pathogen resistance by an external factor, which can be applied at a time point which is most suitable. Thus it prevents unwanted effects, such as for instance can occur due to the relative toxicity of compounds produced during the cascade leading to resistance. Inducible promoters include any promoter capable of increasing the amount of gene product produced by a given gene, in response to exposure to an inducer. In the absence of an inducer the DNA sequence will not be transcribed. Typically, the factor that binds specifically to an inducible promoter to activate transcription is present in an inactive form which is then directly or indirectly converted to the active form by the inducer. The inducer may be a chemical agent such as a protein, metabolite (sugar, alcohol, etc.), a growth regulator, herbicide, or a phenolic compound or a physiological stress imposed directly by heat, salt, wounding, toxic elements etc., or indirectly through the action of a pathogen or disease agent such as a virus. A plant cell containing an inducible promoter may be exposed to an inducer by externally applying the inducer to the cell such as by spraying, watering, heating, or similar methods. Inducible promoters are known to those familiar with the art and several exist that could conceivably be used to drive expression of the genes of the invention. Inducible promoters suitable for use in accordance with the present invention include, but are not limited to, the heat shock promoter, the mammalian steroid receptor system and any chemically inducible promoter. Examples of inducible promoters include the inducible 70 kD heat shock promoter of Drosophila melanogaster (Freeling, M. et al., Ann. Rev. Genet. 19, 297-323) and the alcohol dehydrogenase promoter which is induced by ethanol (Nagao, R. T. et al., in: Miflin, B. J. (ed.) Oxford Surveys of Plant Molecular and Cell Biology, Vol. 3., pp. 384-438, Oxford Univ. Press, 1986). A promoter that is inducible by a simple chemical is particularly useful. Examples for the last category are the promoters described in WO 90/08826, WO 93/21334, WO 93/031294 and WO 96/37609. As examples of a pathogen-inducible promoter the PRP1 promoter (also named gst1 promoter) obtainable from potato (Martini N. et al. (1993), Mol. Gen. Genet. 263, 179-186), the Fis1 promoter (WO 96/34949), the Bet v 1 promoter (Swoboda, I., et al., Plant, Cell and Env. 18, 865-874, 1995), the Vst1 promoter (Fischer, R., Dissertation, Univ. of Hohenheim, 1994; Schubert, R., et al. Plant Mol. Biol. 34, 417-426, 1997), the sesquiterpene cyclase promoter (Yin, S., et al., Plant Physiol. 115, 437-451, 1997) and the gstA1 promoter (Mauch, F. and Dudler, R., Plant Physiol. 102, 1193-1201, 1993) may be mentioned. Also the regulatory region of the ICS gene from Catharanthus roseus, and the promoter region of the proteins MS59 and WL64 (WO 98/13478) may be used in this respect.

The choice of the promoter is not essential, although it must be said that inducible promoters are preferred. It is further known that duplication of certain elements, so-called enhancers, may considerably enhance the expression level of the DNA under its regime (vide for instance: Kay R. et al., Science 236, 1299-1302, 1987: the duplication of the sequence between -343 and -90 of the CaMV 35S promoter increases the activity of that promoter). Also envisaged by the present invention are hybrid promoters, which comprise elements of different promoter regions physically linked.

As regards the necessity of a transcriptional terminator region, it is generally believed that such a region enhances the reliability as well as the efficiency of transcription in plant cells. Use thereof is therefore strongly preferred in the context of the present invention.

As regards the applicability of the invention in different plant species, it has to be mentioned that although a particular embodiment of the invention is merely illustrated with transgenic tobacco plants as an example, the actual applicability in fact not will be limited to these plant species. Any plant species that is subject to some form of pathogen attack, may be transformed with genes according to the invention, allowing the expression products of the gene(s) to trigger the cascade to resistance in some or all of the plant's cells.

Although some of the embodiments of the invention may not be practicable at present, e.g. because some plant species are as yet recalcitrant to genetic transformation, the practicing of the invention in such plant species is merely a matter of time and not a matter of principle, because the amenability to genetic transformation as such is of no relevance to the underlying embodiment of the invention.

Transformation of plant species is now routine for an impressive number of plant species, including both the Dicotyledoneae as well as the Monocotyledoneae. In principle any transformation method may be used to introduce chimeric DNA according to the invention into a suitable ancestor cell, as long as the cells are capable of being regenerated into whole plants. Methods may suitably be selected from the calcium/polyethylene glycol method for protoplasts (Krens, F. A. et al., Nature 296, 72-74, 1982; Negrutiu I. et al,, Plant Mol. Biol. 8, 363-373, 1987), electroporation of protoplasts (Shillito R. D. et al., Bio/Technol. 3, 1099-1102, 1985), microinjection into plant material (Crossway A. et al., Mol. Gen. Genet. 202, 179-185, 1986), DNA (or RNA-coated) particle bombardment of various plant material (Klein T. M. et al., Nature 327, 70, 1987), infection with (non-integrative) viruses and the like. A preferred method according to the invention comprises Agrobacterium-mediated DNA transfer. Especially preferred is the use of the so-called binary vector technology as disclosed in EP A 120 516 and U.S. Pat. No. 4,940,838.

Tomato transformation is preferably done essentially as described by Van Roekel et al. (Plant Cell Rep. 12, 644-647, 1993). Potato transformation is preferably done essentially as described by Hoekema et al. (Hoekema, A. et al., Bio/Technology 7, 273-278, 1989). Generally, after transformation plant cells or cell groupings are selected for the presence of one or more markers which are encoded by plant expressible genes co-transferred with the nucleic acid sequence encoding the protein according to the invention, whereafter the transformed material is regenerated into a whole plant.

Although considered somewhat more recalcitrant towards genetic transformation, monocotyledonous plants are amenable to transformation and fertile transgenic plants can be regenerated from transformed cells or embryos, or other plant material. Presently, preferred methods for transformation of monocots are microprojectile bombardment of embryos, explants or suspension cells, and direct DNA uptake or electroporation (Shimamoto, et al, Nature 338, 274-276, 1989). Transgenic maize plants have been obtained by introducing the Streptomyces hygroscopicus bar-gene, which encodes phosphinothricin acetyltransferase (an enzyme which inactivates the herbicide phosphinothricin), into embryogenic cells of a maize suspension culture by microprojectile bombardment (Gordon-Kamm,, Plant Cell, 2, 603-618, 1990). The introduction of genetic material into aleurone protoplasts of other monocot crops such as wheat and barley has been reported (Lee, Plant Mol. Biol. 13, 21-30, 1989). Wheat plants have been regenerated from embryogenic suspension culture by selecting only the aged compact and nodular embryogenic callus tissues for the establishment of the embryogenic suspension cultures (Vasil, Bio/Technol. 8, 429-434, 1990). The combination with transformation systems for these crops enables the application of the present invention to monocots.

Monocotyledonous plants, including commercially important crops such as rice and corn are also amenable to DNA transfer by Agrobacterium strains (vide WO 94/00977; EP 0 159 418 B1; Gould J, et al., Plant. Physiol. 95, 426-434, 1991).

Following DNA transfer and regeneration, putatively transformed plants may be evaluated, for instance using Southern analysis, for the presence of the chimeric DNA according to the invention, copy number and/or genomic organization. In addition, or alternatively, expression levels of the newly introduced DNA may be undertaken, using Northern and/or Western analysis, techniques well known to persons having ordinary skill in the art. After the initial analysis, which is optional, transformed plants showing the desired copy number and expression level of the newly introduced chimeric DNA according to the invention may be tested for resistance levels against pathogens. Alternatively, the selected plants may be subjected to another round of transformation, for instance to introduce further genes, in order to enhance resistance levels, or broaden the resistance.

Other evaluations may include the testing of pathogen resistance under field conditions, checking fertility, yield, and other characteristics. Such testing is now routinely performed by persons having ordinary skill in the art.

Following such evaluations, the transformed plants may be grown directly, but usually they may be used as parental lines in the breeding of new varieties or in the creation of hybrids and the like.

To obtain transgenic plants capable of constitutively expressing more than one chimeric gene, a number of alternatives are available including the following:

A. The use of DNA, e.g a T-DNA on a binary plasmid, with a number of chimeric genes physically coupled to a selectable marker gene. The advantage of this method is that the chimeric genes are physically coupled and therefore migrate as a single Mendelian locus.

B. Cross-pollination of transgenic plants each already capable of expressing one or more chimeric genes, preferably coupled to a selectable marker gene, with pollen from a transgenic plant which contains one or more chimeric genes coupled to another selectable marker. Afterwards the seed, which is obtained by this crossing, may be selected on the basis of the presence of the two selectable markers, or on the basis of the presence of the chimeric genes themselves. The plants obtained from the selected seeds can afterwards be used for further crossing. In principle the chimeric genes are not on a single locus and the genes may therefore segregate as independent loci.

C. The use of a number of a plurality chimeric DNA molecules, e.g. plasmids, each having one or more chimeric genes and a selectable marker. If the frequency of co-transformation is high, then selection on the basis of only one marker is sufficient. In other cases, the selection on the basis of more than one marker is preferred.

D. Consecutive transformation of transgenic plants already containing a first, second, (etc), chimeric gene with new chimeric DNA, optionally comprising a selectable marker gene. As in method B,the chimeric genes are in principle not on a single locus and the chimeric genes may therefore segregate as independent loci.

E. Combinations of the above mentioned strategies.

The actual strategy may depend on several considerations as may be easily determined such as the purpose of the parental lines (direct growing, use in a breeding programme, use to produce hybrids) but is not critical with respect to the described invention.

In this context it should be emphasised that plants already containing a chimeric DNA capable of triggering the cascade leading to resistance may form a suitable genetic background for introducing further chimeric DNA according to the invention, for instance in order to enhance the speed of the cascade and/or the number of cells which are participating, thereby enhancing resistance levels. The cloning of other genes that can suitably be used in combination with the chimeric DNA, and the obtention of transgenic plants, capable of relatively over-expressing same, as well as the assessment of their effect on pathogen resistance in planta, is now within the scope of the ordinary skilled person in the art.

Plants, or parts thereof according to the invention, including plant varieties, with improved resistance against pathogens may be grown in the field, in the greenhouse, or at home or elsewhere. Plants or edible parts thereof may be used for animal feed or human consumption, or may be processed for food, feed or other purposes in any form of agriculture or industry. Agriculture shall mean to include horticulture, arboriculture, flower culture, and the like. Industries which may benefit from plant material according to the invention include but are not limited to the pharmaceutical industry, the paper and pulp manufacturing industry, sugar manufacturing industry, feed and food industry, enzyme manufacturers and the like.

The advantages of the plants, or parts thereof, according to the invention are the decreased need for biocide treatment, thus lowering costs of material, labour, and environmental pollution, or prolonging shelf-life of products (e.g. fruit, seed, and the like) of such plants. Plants for the purpose of this invention shall mean multicellular organisms capable of photosynthesis, and subject to some form of pathogen attack. They shall at least include angiosperms as well as gymnosperms, monocotyledonous as well as dicotyledonous plants.

The phrase “plants which relatively over-express a chimeric DNA construct” shall mean plants which contain cells expressing a transgene-encoded gene product which is either not naturally present in said plant, or if it is present by virtue of an endogenous gene encoding an identical gene product, not in the same quantity or at the same time, or not in the same cells, compartments of cells, tissues or organs of the plant.

A further aspect of the invention are the regulatory sequences (or promoters) naturally occurring with, operably linked with and regulating expression of the polynucleotides of the invention. It has been found that the expression of the genes harbouring these polynucleotides is found to be starting within a short time delay after application of SA. Under natural circumstances SA is produced upon pathogen infection. Thus, as a result of the inducibility by SA, these regulatory sequences can be said to respond to pathogen attack. The regulatory sequences thus can be used to drive expression of heterologous genes as a response to pathogen infection. Pathogen inducible promoters (such as the prp1-promoter described above) are of great value in biotechnological resistance engineering.

Examples of proteins that may be used in combination with the regulatory regions according to the invention include, but are not limited to, β-1,3-glucanases and chitinases which are obtainable from barley (Swegle M. et al., Plant Mol. Biol. 12, 403-412, 1989; Balance G. M. et al., Can. J. Plant Sci. 56, 459-466, 1976 ; Hoj P. B. et al., FEBS Lett. 230, 67-71, 1988; Hoj P. B. et al., Plant Mol. Biol. 13, 31-42, 1989), bean (Boller T. et al., Planta 157, 22-31, 1983; Broglie K. E. et al., Proc. Natl. Acad. Sci. USA 83, 6820-6824, 1986; Vögeli U. et al., Planta 174, 364-372, 1988); Mauch F. & Staehelin L. A., Plant Cell 1, 447-457, 1989); cucumber (Metraux J. P. & Boller T., Physiol. Mol. Plant Pathol. 28, 161-169, 1986); leek (Spanu P. et al., Planta 177, 447-455, 1989); maize (Nasser W. et al., Plant Mol. Biol. 11, 529-538, 1988), oat (Fink W. et al., Plant Physiol. 88, 270-275, 1988), pea (Mauch F. et al., Plant Physiol. 76, 607-611, 1984; Mauch F. et al., Plant Physiol. 87, 325-333, 1988), poplar (Parsons, T. J. et al., Proc. Natl. Acad. Sci. USA 86, 7895-7899, 1989), potato (Gaynor J. J., Nucl. Acids Res. 16, 5210, 1988; Kombrink E. et al., Proc. Natl. Acad. Sci. USA 85, 782-786, 1988; Laflamme D. and Roxby R., Plant Mol. Biol. 13, 249-250, 1989), tobacco (e.g. Legrand M. et al., Proc. Natl. Acad. Sci. USA 84, 6750-6754, 1987; Shinshi H. et al. Proc. Natl. Acad. Sci. USA 84, 89-93, 1987), tomato (Joosten M. H. A. & De Wit P. J. G. M., Plant Physiol. 89, 945-951, 1989), wheat (Molano J. et al., J. Biol. Chem. 254, 4901-4907, 1979), magainins, lectins, toxins isolated from Bacillus thuringiensis, antifungal proteins isolated from Mirabilis jalapa (EP 0 576 483) and Amaranthus (EP 0 593 501 and U.S. Pat. No. 5,514,779), albumin-type proteins (such as thionine, napin, barley trypsin inhibitor, cereal gliadin and wheat-alpha-amylase, EP 0 602 098), proteins isolated from Raphanus, Brassica, Sinapis, Arabidopsis, Dahlia, Cnicus, Lathyrus and Clitoria (EP 0 603 216), oxalate oxidase (EP 0 636 181 and EP 0 673 416), saccharide oxidase (WO 98/13478), antimicrobial proteins isolated from Allium seeds and proteins from Aralia and Impatiens (WO 95/24485) and the like.

Another use of the inducible promoter is to drive proteins which play a role in the gene-for-gene resistance interaction (e.g. as described in WO 91/15585). Such proteins are, for example, plant proteins such as disclosed in Karrer, E. E. et al. (Plant Mol. Biol. 36, 681-690, 1998), ndr1 and eds1, Cf-proteins and Pto proteins from tomato, the avr-elicitor proteins from Cladosporium fulvum, the avrPto protein from Pseudomonas, and avirulence genes from Xanthomonas, Rhynchosporium secalis and Phytophthora infestans.

However, next to the induction by salicylic acid which is formed endogenously, for example after pathogen infection, it is also possible to induce the regulatory regions by external applications of SA or SA-homologues. This would enable a controlled expression of any gene of interest. In this respect, application of SA or SA-homologues can be in any form, for example as a (foliar) spray or when watering the plants. Preferred SA-homologues to be used are benzoic acid, acetylsalicylic acid, polyacrylic acid and substituted derivatives thereof and homologues based on the benzo-1,2,3-thiadiazole structure and include, but are not limited to, the following types of compounds: benzo-1,2,3-thiadiazolecarboxylic acid, benzo-1,2,3-thiadiazolethiocarboxylic acid, cyanobenzo-1,2,3-thiadiazole, benzo-1,2,3-thiadiazolecarboxylic acid amide, and benzo-1,2,3-thiadiazolecarboxylic acid hydrazide. Specifically preferred are benzo-1,2,3-thiadiazole-7-carboxylic acid, methyl benzo-1,2,3-thiadiazole-7-carboxylate, n-propyl benzo-1,2,3-thiadiazole-7-carboxylate, benzyl benzo-1,2,3-thiadiazole-7-carboxylate, benzo-1,2,3-thiadiazole-7-carboxylic acid sec-butylhydrazide, 2,6-dichloroisonicotinic acid, or methyl 2,6-dichloroisonicotinate.

The following state of the art may be taken into consideration, especially as illustrating the general level of skill in the art to which this invention pertains. EP-A 392 225 A2; EP-A 440 304 A1; EP-A 460 753 A2; W090/07001 A1; U.S. Pat. No. 4,940,840.

Evaluation of Transgenic Plants

Subsequently transformed plants are evaluated for the presence of the desired properties and/or the extent to which the desired properties are expressed. A first evaluation may include the level of expression of the newly introduced genes, the level of induction of pathogen-related or other proteins, the pathogen resistance of the transformed plants, stable heritability of the desired properties, field trials and the like.

Secondly, if desirable, the transformed plants can be crossbred with other varieties, for instance varieties of higher commercial value or varieties in which other desired characteristics have already been introduced, or used for the creation of hybrid seeds, or be subject to another round of transformation and the like.

The present invention will now be further described by way of the following non limiting examples in conjunction with the above described figures and the sequence listing of which:

SEQ ID No. 1-9=Polynucleotides which encode proteins which are naturally expressed upon induction of salicylic acid.

SEQ ID No. 10=Polynucleotide which encodes a protein which is naturally expressed upon induction of salicylic acid and promoter sequence thereof.

SEQ ID No. 11=Protein sequence of SEQ ID No. 10.

SEQ ID No. 12-14=PCR Primers.

SEQ ID Nos. 15-33=Primers AP1 to AP 19 respectively.

### EXAMPLE 1

In our efforts to identify immediate early SA-induced genes we have used the following observations. A sequence element in the 35S enhancer responsible for SA induction appears to be the activation sequence 1 (as-1). Distinctive features of activation through as-1 are that (1) induction occurs within 30 minutes, (2) mRNA levels peak in 1-4 h and then decline, (3) induction occurs in the presence of cycloheximide (CHX), (4) co-treatment with CHX and SA produces a continuous accumulation of mRNA beyond 4 h. The induction mechanism is typical of viral and cellular immediate early- or primary-reponse genes (Herschman (1991) Annu. Rev. Biochem 60, 281-319), such as the response of animal cells to β and γ-interferon (Larner et al. (1986) J. Biol. Chem. 261, 453-459; Lew et al. (1989) Mol. Cell Biol. 9, 5404), and the induction of certain auxin-induced genes (reviewed in Abel and Theologis (1996)Plant Phys. 111, 9-17).

Differential display technology (Liang, P. and Pardee, A. B., Science 257: 967-971, 1992) was used to identify genes activated in the presence of SA, CHX and both. Therefore the absence or presence of SA, induction at 3 timepoints of SA-treatment, 2 different concentrations of SA and the effect of addition of CHX were measured. Seven treatments in total were performed. These are listed in the table below. Treatment of tobacco BY-2 suspension cells and leaves and differential display PCR reactions were performed as described (Horvath and Chua (1996), Plant Mol. Biol. 31, 1061-1072).

| Treatment | Chemicals added/time |
|---|---|
| 1 | H₂O |
| 2 | 200 μM SA, 4 hrs |
| 3 | 71 μM CHX, 5 hrs |
| 4 | 71 μM CHX, 5 hrs + 200 μM SA after 1 hr (so 4 hrs total) |
| 5 | 20 μM SA, 2 hrs |
| 6 | 200 μM SA, 2 hrs |
| 7 | 200 μM SA, 8 hrs |

Thirty eight reactions were carried out using downstream primers T12MG or T12MC and upstream primers AP1-19 (SEQ ID NO's: 15-33). The nomenclature for each differentially expressed PCR product is derived from the primer combination that was used for its amplification (e.g. C6-2 is the second band of interest arising from PCR using primers T12MC and AP6).

From the 38 sets of reactions, some 60 differentially displayed products with an interesting expression pattern were identified. Fiftytwo were recovered for testing on poly(A)+Northern blots loaded with mRNA derived from cells from each of the treatments.

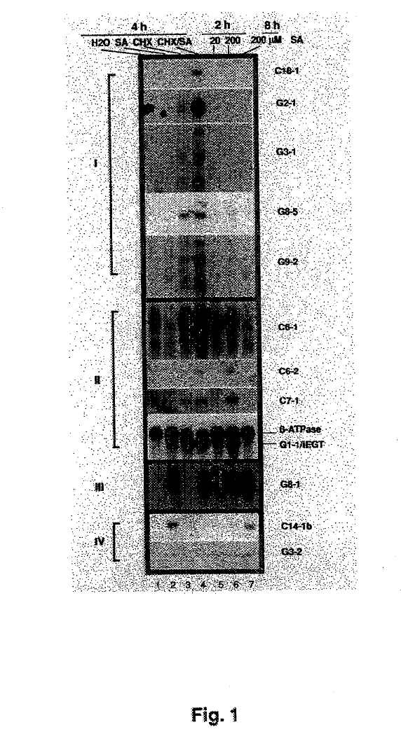

About fifteen showed interesting mRNA expression patterns on the Northern blot and 12 of these are shown in FIG. 1. The remaining three (C3-2, C16-1 and C2-1) produced distinctive yet weak signals and were not pursued further.

### EXAMPLE 2

From the twelve differentially expressed transcripts, nine were subcloned, sequenced, and their expression patterns rechecked. (SEQIDNO: 1-9).

Based on different kinetics of induction these transcripts may be classified in 4 categories (see also the summary in Table 2).

Class I Response Genes

Class I genes (C3-2, C16-1 (SEQ ID NO: 7), C18-1 (SEQ ID NO: 1), G2-1 (SEQ ID NO: 6), G3-1 (SEQ ID NO: 9), G8-5 (SEQ ID NO: 2), G9-2) are characterized by (1) little if any detectable mRNA accumulation following SA-treatment at 2,4 or 8 h, (2) some induction by CHX treatment alone, and (3) a much greater induction by CHX and SA co-treatment. Superinduction by CHX is a phenomenon widely observed with mammalian immediate-early (IE) genes (Almendral, J. M. et al., Mol. Cell. Biol. 215: 403-410, 1988; Zipfel, P. F. et al., Mol. Cell. Biol. 9: 1041-1048, 1989). This suggests that this class of proteins may have an early function in mediating the effect of SA.

Class II Response Genes

Exemplified by C2-1 (SEQ ID NO: 4), C6-1, C6-2 (SEQ ID NO: 3), C7-1 and G1-1, class II genes are characterised by rapid and transient SA induction, with mRNA accumulation at the earliest (2 h) timepoint, and decreased levels at later timepoints (4 and 8 h). CHX also induced expression of class II genes and CHX/SA co-treatment produced similar or somewhat greater levels of steady state mRNA. Class I and class II genes are similar in their behaviour except that overall mRNA accumulation is greater with class II genes, whereas there was a massive accumulation of class I mRNAs following CXH/SA co-treatment. Similar mechanisms of induction may apply to regulation of class I and class II genes, but there may be additional levels of regulation for the class I genes to maintain low abundance.

TABLE 2
Summary of early SA-induced genes identified by differential display.

| ddPCR | Response | Fragment size | |
|---|---|---|---|
| product | class | (bp) | Approx. mRNA size(nt)¹ |
| C3-2 | I | 190 | 6000/2800/850 |
| C16-1 | I | 203 | 5000/4000² |
| C18-1 | I | 426 | 1200 |
| G2-1 | I | 524 | 520 |
| G3-1 | I | 276 | 2000/1000/700 |
| G8-5 | I | 437 | 1500 |
| G9-2 | I | 230 | 4000/2000/1600 |
| C2-1 | II | 382 | 5-6000 |
| C6-1 | II | 430 | 2000/1300 |
| C6-2 | II | 174 | 1300/1000 |
| C7-1 | II | 430 | 800 |
| G1-1 | II | 297 | 1600 |
| G8-1 | III | 400 | 550 |
| C14-1b | IV | 298 | 1000 |
| G3-2 | IV | 282 | 5000 |

¹Slashes separate multiple messagers of different sizes
²The two transcripts detected with the cloned ddPCR product had differential patterns of expression and may represent related sequences encoded by different genes.

Class III Response Genes

Class III is represented by a single member G8-1, which showed rapid mRNA induction by SA (at 2 h), that was sustained through 4 and 8 h. CHX alone did not induce G8-1 and the presence of CHX did not significantly alter induction by SA.

Class IV Response Genes

Similar to class III, class IV genes (G3-2 (SEQ ID NO: 5) and C14-1b (SEQ ID NO: 8)) showed rapid and sustained induction upon SA treatment, with elevated levels at 2, 4 and 8 h. CHX did not only fail to induce class IV genes, but also blocked the induction by SA.

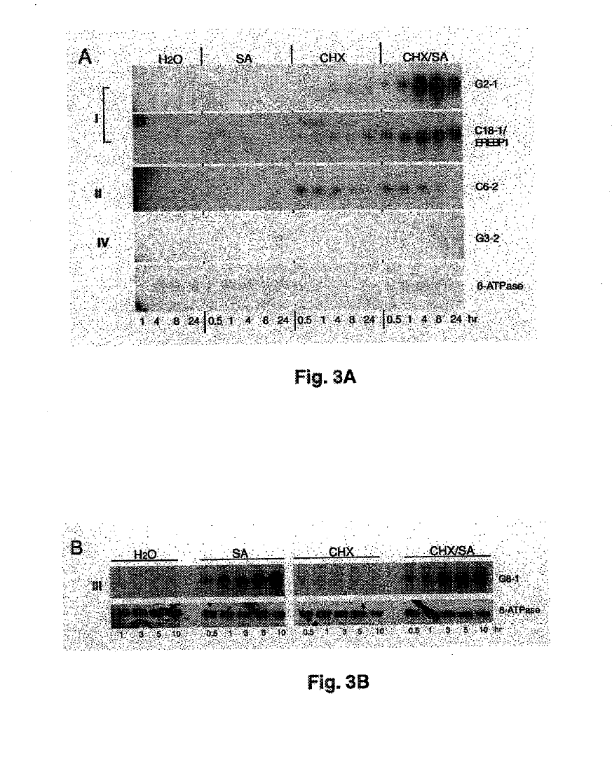

Overall, most of the genes had comparable sensitivity to SA, with a clear induction at 200 μM SA, and little or none at 20 μM SA. Two genes, G8-1 and G3-2 demonstrated greater sensitivity to SA, with 20 μM producing mRNA levels comparable to those at 200 μM. A more detailed analysis of expression pattern in time for representative candidates of each of the classes is shown in FIGS. 3A and B.

### EXAMPLE 3

Sequence analysis of the available fragments revealed that many of the genes shared no homology to database sequences. These genes may therefore represent previously unrecognized loci, or the PCR fragments were of insufficient length to reveal a coding region homology. Four sequences did produce noteworthy matches.

G1-1 was found to be similar to plant flavonoid glucosyltransferase, and was analyzed before (Horvath, D. M. and Chua, N.-H., Plant Mol. Biol. 31: 1061-1072, 1996).

Clone C16-1 was found to have similarity to several phosphorylase kinases, suggesting it may play a role in SA signal transduction. Clone G8-5 was found to have a strong similarity to an Arabidopsis retrotransposon (Bevan et al., EU sequencing project, 1997), and to the endonuclease/integrase region of the maize retrotransposon Hopscotch polyprotein (White, S. E. et al., Proc. Natl. Acad. Sci., USA 91: 11792-11796, 1994), but the homology was found exclusively in the minus strand of G8-5.

A perfect match was found between C18-1 and the previously cloned tobacco gene Ethylene Reponsive Element Binding Protein-1 (EREBP1) (Ohme-Takagi, M. and Shinshi, H., Plant Cell 7: 173-182, 1995).

### EXAMPLE 4

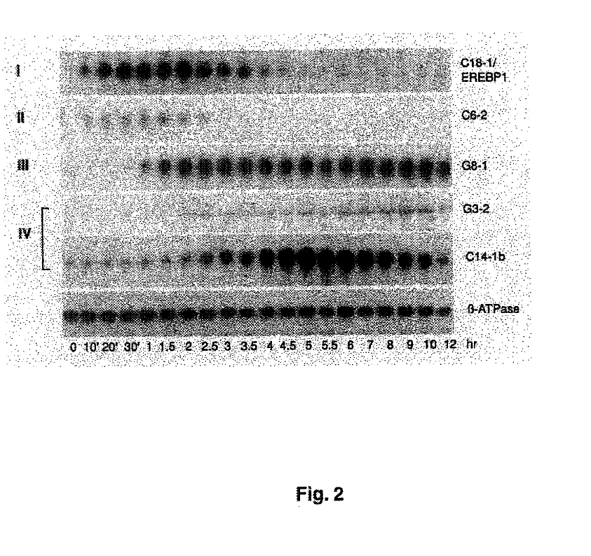

The kinetics of induction were examined in detail through time course experiments for representative clones of each class (FIG. 2). Although no SA-induced signal was present on poly(A)+mRNA gel blots using the ddPCR fragment for any class I gene, enhanced conditions using the full length cDNA of C18-1/EREBP1 for probe made it possible to visualize a signal. In fact, SA did induce a very rapid and transient increase in C18-1/EREBP1 mRNA levels, first apparent in 10 minutes, peaking at 2 h and decaying rapidly to background levels by 4.5 h.

The class II gene C6-2 demonstrated similar kinetics, with induction apparent within 10 minutes, peak at approximately 1.5 h, and reduction to background levels by 4 h. Previously we have shown another class II gene G1-1/EGT (Horvath and Chua, 1996), which was induced slighly later at 20-30 minutes and reached a peak at 3 h.

The class III gene G8-1 was first detected between 30-60 minutes, and reached a plateau at 2-2.5 h that maintained for at least 12 h. G3-2, a class IV gene was not detectably activated prior to 2 h, but between 2 and 12 h mRNA levels increased steadily. Another class IV gene C14-1b, demonstrated a slightly different response profile. Induction was apparent at 2-2.5 h, but expression in this case peaked at 5 h and showed a steady decline thereafter.

The rapid and transient reponses of class I and II genes were most similar to those identified in mammalian IE gene responses, whereas class III and IV, though expressed early, appeared to be regulated by a different mechanism.

### EXAMPLE 5

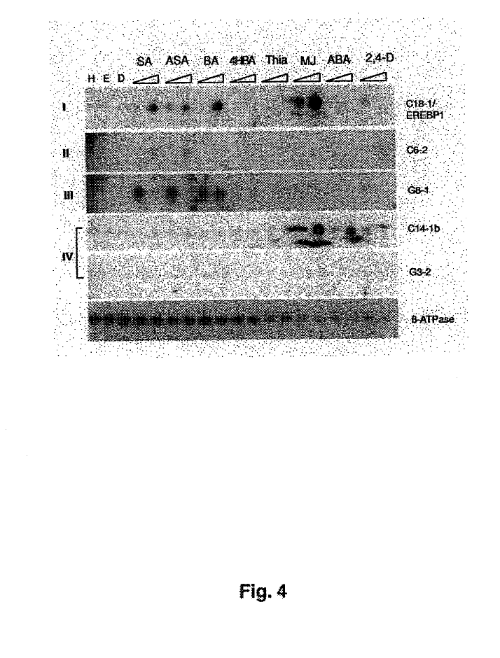

The specificity for induction by SA was tested by treating tobacco cells with a range of SA analogs and plant signalling compounds at 0.1 and 1 mM for approximately 2.5 h. The results in FIG. 4 demonstrate a wide range in the responses of individual genes. Among the SA-analogs, acetyl-SA (ASA, aspirin), benzoic acid (BA) and 4-hydroxybenzoic acid (4HBA), responses generally reflected the ability of the analog to substitute for SA in other cellular responses (Yalpani, N. et al., Plant Cell 3: 809-818, 1991, Yang, Y. and Klessig, D. F., Proc. Natl. Acad. Sci. 93, 14972-14977, 1996). ASA, typically as effective as SA, induced all genes to similar levels as SA. BA, a partially active analog, also induced the expression, and the inactive analog 4HBA failed to induce all genes but C6-2. An interesting distinction among the responses is that G8-1 and G3-2 both showed a high sensitivity to SA, ASA and BA, showing equal or greater induction at 0.1 mM versus 1 mM, whereas C18-1/EREBP1 and C6-2 showed greater induction at 1mM. C14-1b did not reveal response to SA or analogs in this time frame. All other genes displayed specificity for induction by active SA-analogs, except for C6-2, which may respond with the behavior of an electrophile responsive gene (Ulmasov, T. et al., Plant Mol. Biol. 26: 1055-1064, 1994). Among the other components tested, thiamine, a strong inducer of PR genes (Asselin, A. et al., Can. J. Bot. 63: 1276-1282, 1985) did not produce a response from any of the genes tested. Methyljasmonate (MJ), a plant compound involved in wound responses was able to induce expression of several genes, including C18-1/EREBP1, C14-1b and G3-2. In fact, MJ was the most effective inducer for C18-1/EREBP1 and C14-1b. Interestingly, C14-1b was the only gene that could be induced by ABA, a signal molecule involved in wound responses and drought and cold stress. Several genes, including those for proteinase inhibitors and lipoxygenase (Hildmann, T. et al., Plant Cell 4: 1157-1170, 1992; Melan, M. A. et al., Plant Physiol. 101: 441-450, 1993; Xu, D. et al., Plant Mol. Biol. 22: 573-588, 1993) are known to be under dual regulation by MJ and ABA. A similar mode of regulation may be in effect with C14-1b. the auxin 2,4-D induced several genes including C18-1/EREBP1 and C6-2. Thus although several of the ddPCR gene displayed a trend towards activation by active SA analogs, all but G8-1 were significantly induced by additional plant compounds, suggesting additional roles for these genes in other response pathways.

### EXAMPLE 6

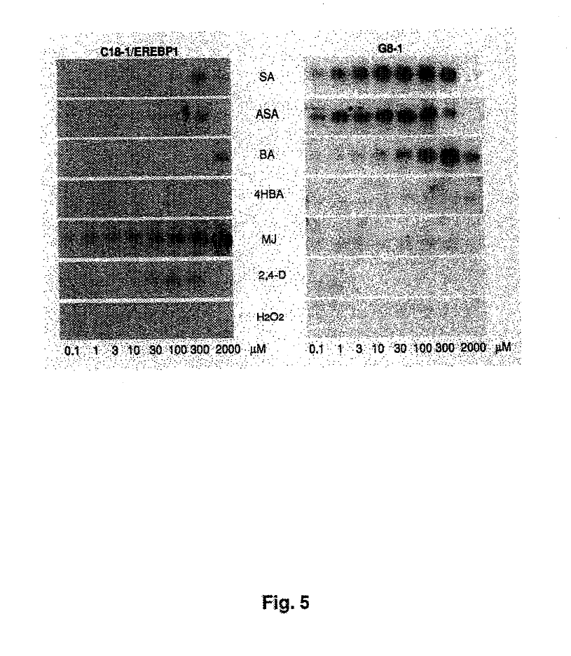

A dose-response curve was made for SA. Therefore tobacco cells were treated with SA in a range of concentrations from 0.1 μM to 2 mM. The results are shown in FIG. 5.

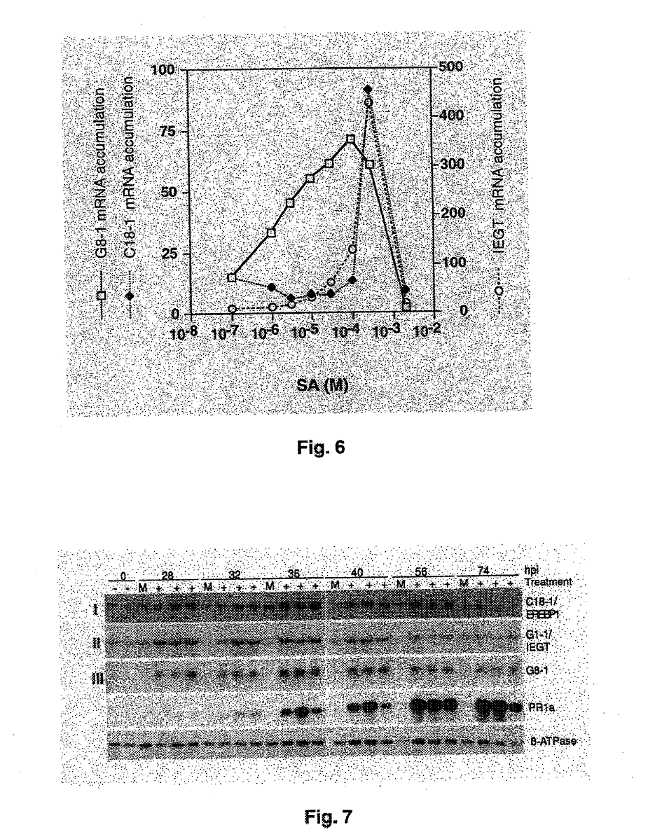

There is a clear difference in sensitivity between e.g. C18-1/EREBP1 and G8-1. C18-1/EREBP1 has a narrow response zone, whereas G8-1 expression is activated over a large SA concentration range (see FIG. 6).

The concentration of SA required for half of the maximum level of C18-1/EREBP1 expression is 100-150 μM, whereas for G8-1 this value is approximately 1 μM. G3-2 seems to be similarly sensitive as G8-1 (data not shown). Data for another SA-inducible gene (IEGT; Horvath and Chua, 1996) are shown as well in this graph.

### EXAMPLE 7

Pathogen-responsiveness of the promoters was tested in a TMV-induced HR reaction in tobacco leaves. Results are shown in FIG. 7. Both C18-1/EREBP1 (Class I) and G1-1 (Class II) were expressed at higher levels in TMV-infected tobacco leaves than in control plants between 28 and 56 h. The kinetics of activation closely follow the timing of induced SA biosynthesis, which begins at between 24-36 hpi (Malamy, J. et al., Science 250, 1002-1004, 1990). By 74 h post TMV infection, however, mRNA levels from both genes had returned to basal levels equivalent to those in control plants.

Levels of another SA-induced gene (PR1a) were induced later and were more sustained.

### EXAMPLE 8

Genomic clones of IEGT, G8-1 and C18-1/EREBP1 were isolated from a tobacco genomic library (in lambda EMBL3) purchased from Clontech (Palo Alto, Calif.).

A 2.1 kb BamHI-SalI genomic fragment (SEQ ID NO: 10) containing the IEGT sequence (labelled IS5a) was cloned into vector pBSK (Stratagene, La Jolla, CA). The isolated fragment consists of 524 bp of upstream sequence (5′ UTR and promoter sequences), 1431 bp of ORF, with no apparent introns and 220 bp of sequence downstream of the coding sequence.

The promoter and 5′ untranslated sequence region of the genomic clone, were amplified by PCR. Downstream primers were designed to prime at 1) the presumed start of transcription and 2) at the start of the Open Reading Frame (ORF) to include the 5′ UTR. The sequences of the primers used are SEQIDNO: 12, 13 and 14.

Amplified fragments were digested with BamHI and HindIII and subcloned into BamHI and HindIII-digested vector VIP11•-FFLuc (Anderson et al., 1994, Plant J. 4, 457-470) containing the firefly luciferase gene. This vector positions the promoter sequences directly upstream the promoterless FFLuc gene. Construct pIEGT-Luc1 contains the smaller promoter fragment (432 bp), lacking the 5′ UTR sequences, whereas pIEGT2-Luc2 consisted of the promoter plus 5′ UTR sequences.

Both luciferase constructs were transformed to tobacco using Agrobacterium-mediated transformation.

### EXAMPLE 9

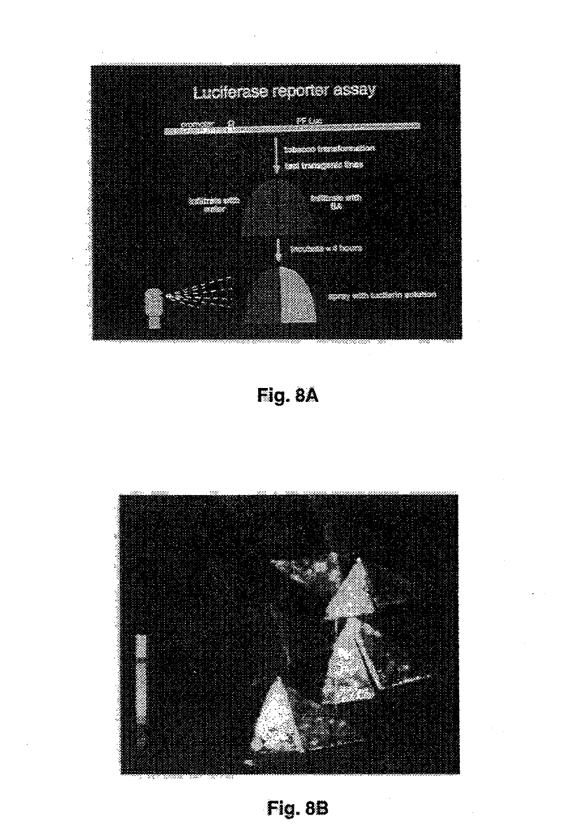

Leaves of primary transformants or S1 progeny were tested for reporter gene activity by excising halves of the leaves, splitting and infiltration with either water or 1 mM SA for 4-12 hours. Leaves were then sprayed with the reporter substrate luciferin (1 mM luciferin with 0.1% Triton X-100 in water) and the luminescence counted by video-imaging and quantitation equipment (schematically shown in FIG. 8A). Six out of ten IEGT-Luc1 lines showed SA-induced luciferase expression that ranged from 2.3-6 fold over controls (see FIG. 8B). All ten IEGT-Luc2 lines tested showed only baseline activity, regardless of treatment.

Challenge with pathogens was performed by inoculating leaves with Tobacco Mosaic Virus (TMV). Since the tobacco plants carry the N gene they are resistant to TMV infection. NN tobacco induces SA biosynthesis, defense responses and cell death upon challenge with TMV. IEGT-luc1 lines showed a clear induction of luciferase expression over time, when assayed at 21 and 52 h post inoculation.

### EXAMPLE 10

Construction of a SA-inducible promoter insecticidal protein chimeric construct

The SA-inducible promoters (like the promoter sequence 1-431 of SEQ ID NO: 10) are coupled to the coding region of insecticidal proteins like the Bacillus thuringiensis-derived Cry genes (as an example the genes described in U.S. Pat. No. 5,635,480 and/or EP application 86300291.1 can be used). To this end, the Open Reading Frame of the insecticidal protein is functionally linked to the transcriptional regulatory fragment of the SA-inducible promoter. Ideally, all of the upstream regulatory sequences of the SA-responsive gene are used up to the translation start codon, at which point the Open Reading Frame encoding the insecticidal protein starts. Terminator/polyadenylation sequences are added after the Open Reading Frame of the insecticidal protein to enhance expression of the gene. Constructs thus made are transformed to plants using standard protocols.

Expression of the insecticidal protein is measured before and after SA- or SA analogue-treatment of the plants using ELISA, Western blot analysis or biological assays.

Plants that express high levels of insecticidal protein are tested under fieldconditions for increased resistance to target insects after treatment with SA or an SA-analogue.

Expression from the SA-inducible promoters can be enhanced, if so desired, using a number of well-described techniques. One of these comprises multimerisation of part of the transcription-activating sequences of that promoter, or using combinations of SA-inducible promoters (see e.g. EP 0729514) or by linking strong constitutive enhancers upstream of the SA-regulatory region (see e.g. Ph. D. Thesis Regina Fischer, Universitaet Hohenheim 1994 for similar work enhancing expression from a pathogen-responsive gene).

1. A polynucleotide which when operably linked to its native regulatory sequence is capable of producing a protein upon induction with salicylic acid characterised in that the said polynucleotide comprises a sequence which is selected from the group essentially consisting of SEQ ID Nos. 1 to 9 or is a polynucleotide sequence which encodes the amino acid sequence of SEQ ID No. 11.

2. A chimeric DNA sequence comprising a nucleotide sequence according to claim 1.

3. A chimeric DNA sequence according to claim 2, further comprising a transcriptional initiation region and, optionally, a transcriptional termination region.

4. A chimeric DNA sequence according to claim 3 wherein the transcriptional initiation region is an inducible promoter.

5. A chimeric DNA sequence according to claim 4, characterized in that the inducible promoter is a pathogen inducible promoter.

6. A chimeric DNA sequence according to claim 4, characterized in that the inducible promoter is a chemically inducible promoter.

7. A chimeric DNA sequence according to any of claims 2-6 which is a vector.

8. A host cell comprising a vector according to claim 7 and which is capable of maintaining said vector once present therein.

9. A host cell stably incorporating in its genome a nucleotide sequence according to claim 1.

10. A promoter characterised in that it comprises the nucleic acid sequence naturally occurring 5′ and capable of regulating the transcription of the polynucleotide or polynucleotide sequence according to claim 1.

11. A pathogen inducible promoter, characterized in that it comprises nucleotides 1-431 of SEQ ID NO: 10.

12. A vector comprising a pathogen inducible promoter according to claim 10 or 11.

13. A host cell comprising a vector according to claim 12.

14. A host cell according to any of claims 8, 9 or 13 which is a plant cell.

15. A plant or a plant part comprising at least one plant cell according to claim 14.

16. A plant or a plant part consisting essentially of plant cells according to claim 14.

17. A method to make plants resistant to pathogen attack, characterized in that they are transformed with a vector comprising a nucleic acid sequence according to claim 1.

18. A method for expressing proteins in plants characterised in that the promoter according to claim 10 or claim 11 is used as the regulatory region which is operably linked to a polynucleotide encoding the said protein to be expressed.

19. A method according to claim 18 wherein the said promoter is induced by salicylic acid or a homologue thereof.

20. A method according to claim 18 wherein the promoter is induced by a pathogen infection.
